(19) United States
(12) Patent Application Publication
(10) Pub. No.: US 2011/0009907 A1
(43) Pub. Date: Jan. 13, 2011

(54) TRANS-PEDICULAR INTERBODY FUSION

(76) Inventor: Assaf Klein, Kibbutz Hama'apil (IL)

Correspondence Address: David Klein;DEKEL PATENT LTD., Beit HaRof'im, 18 Menuha VeNahala Street, Room 27, REHOVOT, IL

(21) Appl. No.: 12/499,808

(22) Filed: Jul. 9, 2009

(51) Int. Cl. A61B 17/88

(52) U.S. Cl. 606/279

(57) ABSTRACT

A method including affixing an anterior mechanical supportive fastener to anterior portions of adjacent spinal structure by introducing the fastener from a posterior portion of a vertebra and passing the fastener through a curved portion to the anterior portions and fastening the fastener to the anterior portions.

### FIELD OF THE INVENTION

The present invention relates to a method for spinal fixation of vertebral structure from a posterior direction through pedicles.

### BACKGROUND OF THE INVENTION

A number of conditions can result in damage or deterioration of one or more intervertebral discs of the human spine. For example, a disc may become damaged by external injury or degenerative disc disease due to advanced age in combination with other factors.

One type of surgery which has benefitted patients with disc problems is fusion of the vertebrae adjacent the disc. In this surgery, the natural disc is replaced with a spacer and the vertebrae are fixed relative to one another.

There are several approaches to insert this spacer: anterior, posterior, posterior lateral, lateral, oblique or axial approaches. The anterior approach can be dangerous because it necessitates surgical involvement of the two major blood vessels, the vena cava and the aorta. Anterior insertion requires spreading the abdominal muscles and the aorta and vena cava in order to gain access to the vertebrae and the disc. Because rupture of either of the vena cava or aorta is life-threatening, such surgery requires the attendance of both a vascular surgeon as well as a spinal surgeon. The posterior, posterior lateral, and lateral approaches involve the risk of nerve impingement that may cause neural damage and even paralysis. The oblique approach requires very precise insertion that involves the risk of pedicle violation, and unlike aspects of the invention described hereinbelow, does not allow 360 degree fixation combined with a pedicle-screw-based posterior fixation system. The axial approach is limited to the S1-L5 pathologies.

In certain situations, it would be desirable to have stiffening, stabilization or spinal fixation systems for the spinal column that include intervertebral plates or spacers as well as pedicle screws fixed in the vertebrae and connected to each other via a connecting rod, such that the distances and arrangement of the vertebrae can be aligned and fixed relative to each other. This would provide a more rigid construct, and reduce moment loads transfer to the vertebrae and the instrumentation.

### SUMMARY OF THE INVENTION

The present invention seeks to provide a spinal fixation system, described more in detail hereinbelow, wherein a mechanical supportive fastener is used to fix adjacent vertebrae and is introduced from a posterior entry path through the pedicles as for pedicle screws.

The mechanical supportive fastener may be, without limitation, a threaded fastener (e.g., a screw), an expandable fastener, or an injectable fastener. In one embodiment of the invention, the mechanical supportive fastener is used independently of any other fastening device, and is implanted with a dedicated instrumentation/delivery system. In another embodiment of the invention, the mechanical supportive fastener is combined with posterior lateral pedicle screws fixed together with bars or rods (posterior lateral fixation—PLF) to provide a 360 degree fixation system.

Any of the mechanical supportive fasteners of the invention (e.g., screw, expandable and injectable) can be installed as single device through one pedicle in a central portion of the spinal structure, or as a symmetric two-sided system through two pedicles. The system of the invention can be installed through the pedicles of the superior or the inferior levels (e.g., through the L4 pedicles or L5 pedicles in L4-5 fusion).

In another embodiment of the invention, the mechanical fastener is connected at the adjacent level to an anterior fastener, which provides a much more rigid construct in PLF (for example, double locking of the mechanical fastener with the pedicle screws).

There is provided in accordance with a non-limiting embodiment of the present invention a method including affixing an anterior mechanical supportive fastener to anterior portions of adjacent spinal structure by introducing the fastener from a posterior portion of a vertebra and passing the fastener through a curved path to the anterior portions and fastening the fastener to the anterior portions. The fastener may be introduced through a pedicle at the posterior portion of the vertebra.

In accordance with one embodiment of the present invention the fastener is a threaded fastener and fastening the fastener includes turning the fastener to tighten the fastener.

In accordance with another embodiment of the present invention the fastener is an expandable fastener having an expandable portion and fastening the fastener includes turning the fastener to expand the expandable portion outwards.

In accordance with yet another embodiment of the present invention the fastener is an injectable fastener having an expandable portion that is fillable and expandable outwards with a fluid and fastening the fastener includes injecting fluid into the expandable portion to expand the expandable portion outwards.

In accordance with an embodiment of the present invention pedicle screws are affixed in at least one of the adjacent vertebrae, and a connecting rod is secured between heads of the pedicle screws.

In accordance with an embodiment of the present invention the threaded fastener is fastened with a flexible screwdriver. Alternatively, the threaded fastener is fastened with a flexible shaft, which serves as an elastic joint for transferring turning torque to the fastener.

In accordance with an embodiment of the present invention the method further includes connecting the fastener to the pedicle screw.

### BRIEF DESCRIPTION OF THE DRAWINGS

The present invention will be understood and appreciated more fully from the following detailed description taken in conjunction with the drawings in which:

FIGS. 1, 2 and 3 are simplified pictorial illustrations of a method for fixation of vertebral structure from a posterior direction through the pedicles, in accordance with an embodiment of the present invention, using threaded fasteners;

FIGS. 4 and 5 are simplified pictorial illustrations of a method for fixation of vertebral structure from a posterior direction through the pedicles, in accordance with another embodiment of the present invention, using expandable fasteners;

FIGS. 6 and 7 are simplified pictorial illustrations of a method for fixation of vertebral structure from a posterior direction through the pedicles, in accordance with yet another embodiment of the present invention, using injectable fasteners; and

FIG. 8 is a simplified pictorial illustration of a method for fixation of vertebral structure from a posterior direction through the pedicles, in accordance with still another embodiment of the present invention, using a mechanical fastener connected at the adjacent level to an anterior fastener.

### DETAILED DESCRIPTION OF THE PREFERRED EMBODIMENT

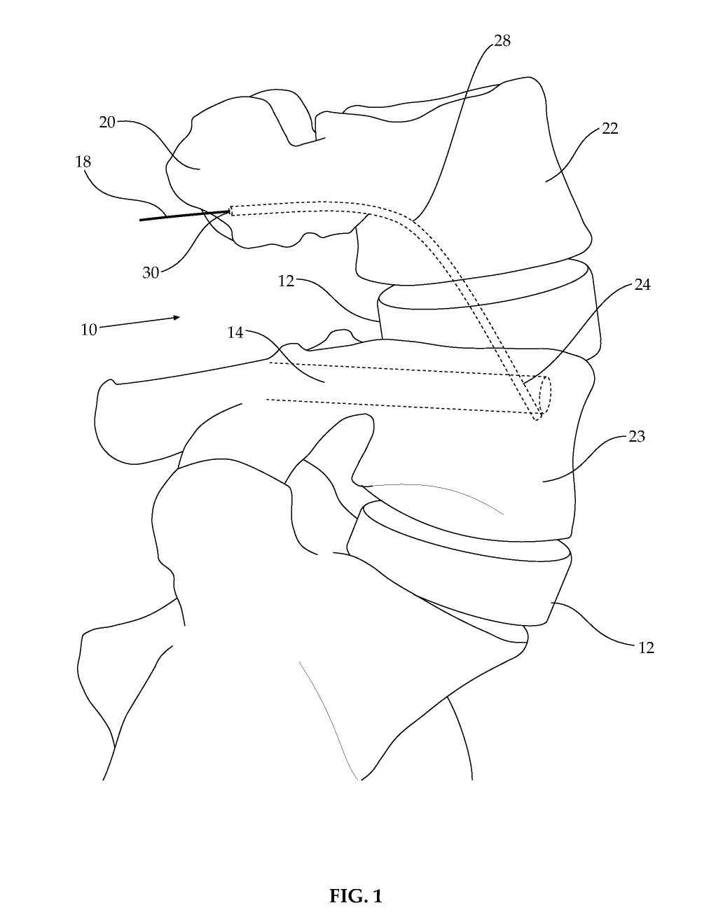

Reference is now made to FIGS. 1-3, which illustrate a method for fixation of vertebral structure from a posterior direction through the pedicles, in accordance with a non-limiting embodiment of the present invention.

FIGS. 1-3 illustrate a spinal fixation system 10 (also alternatively referred to as a stiffening or stabilization system), that employs a mechanical supportive fastener for fixation of vertebral structure, such as a fastener fixed between endplates of two adjacent vertebrae 22 and 23, and which may pass through intervertebral disc 12, or other spinal structure. Alternatively, the disc 12 shown in the figures may be an artificial anterior support or device installed in an anterior portion of the vertebrae. The use of the artificial support depends on the patient and situation.

The mechanical supportive fastener is referred to as an anterior mechanical supportive fastener because it fastens natural and/or artificial spinal structure located at anterior portions of the vertebrae, but the fastener is introduced from a posterior location.

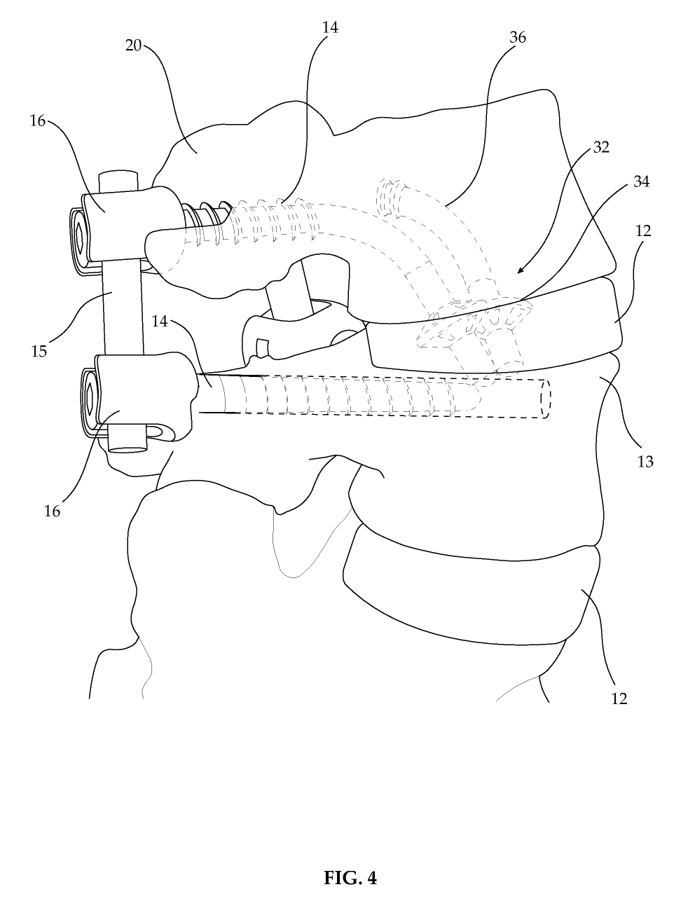

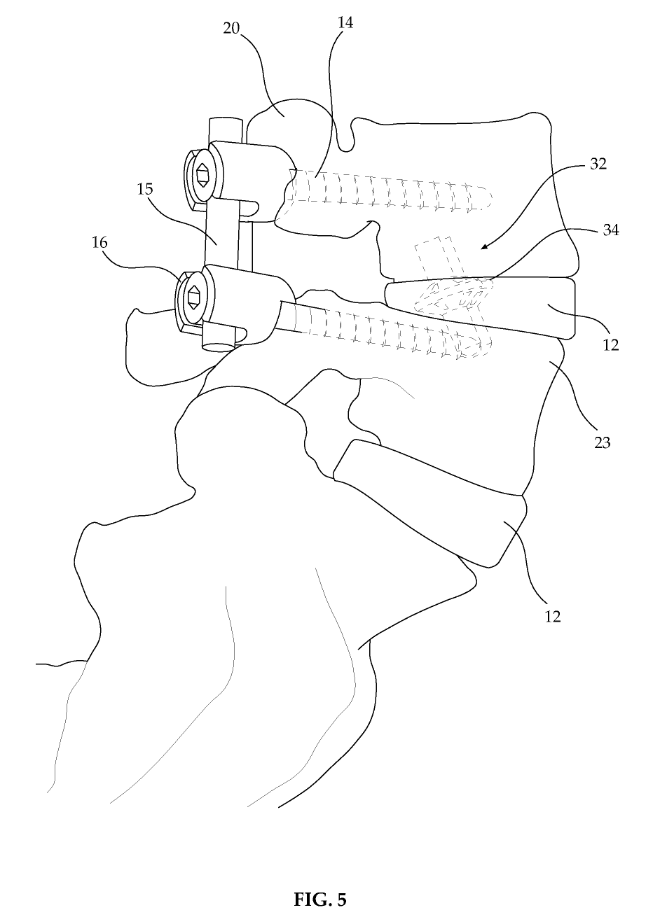

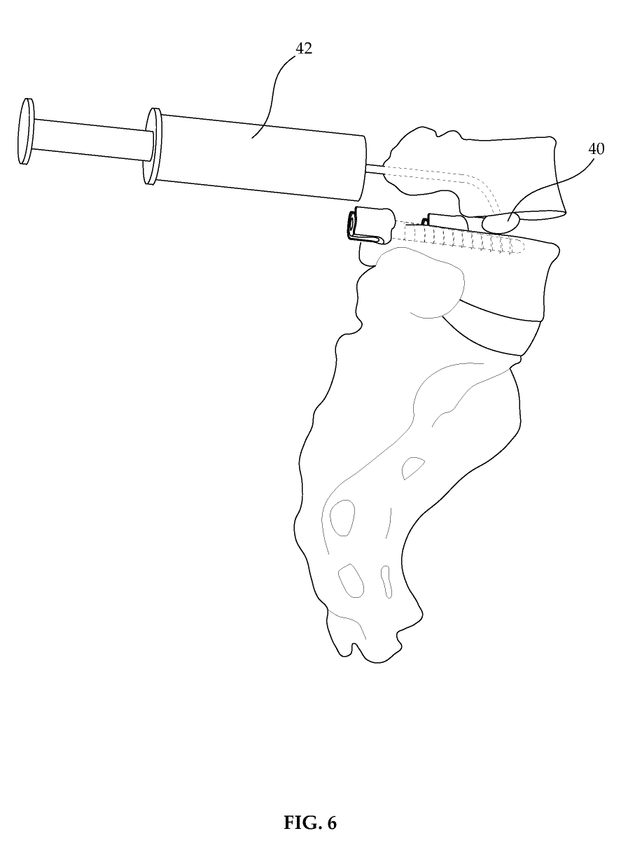

In accordance with an embodiment of the present invention, the anterior mechanical supportive fastener is affixed through the same entry point as a pedicle screw 14. This avoids the dangerous anterior access to the vertebrae and the disc, with no risk for nerve root impingement or damage to the vena cava or aorta. In the embodiment of FIGS. 1-3 the anterior mechanical supportive fastener is a threaded fastener; in the embodiment of FIGS. 4-5 the anterior mechanical supportive fastener is an expandable fastener; and in the embodiment of FIG. 6 the anterior mechanical supportive fastener is an injectable fastener, as is explained below.

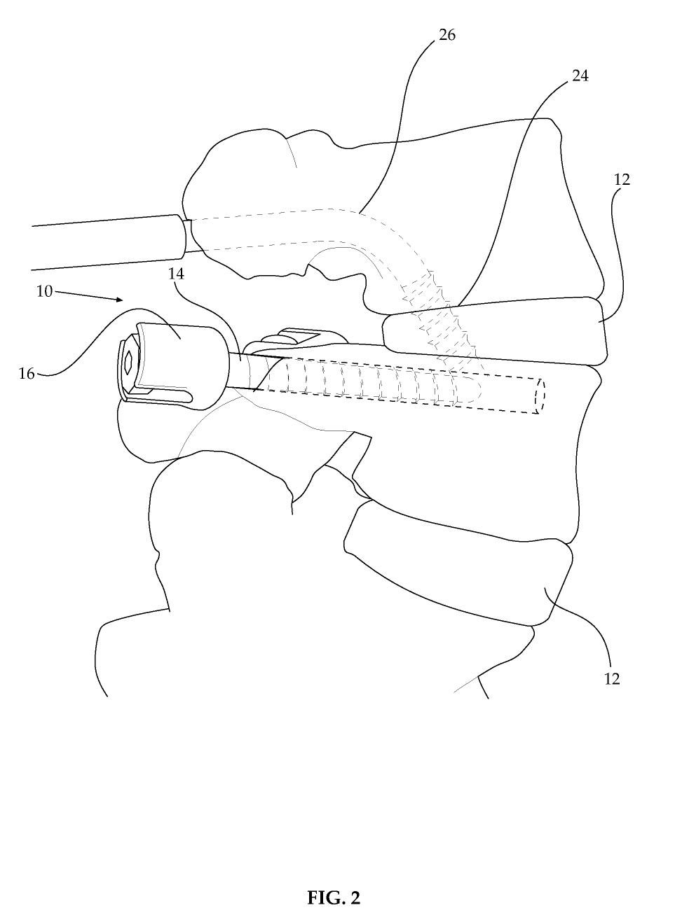

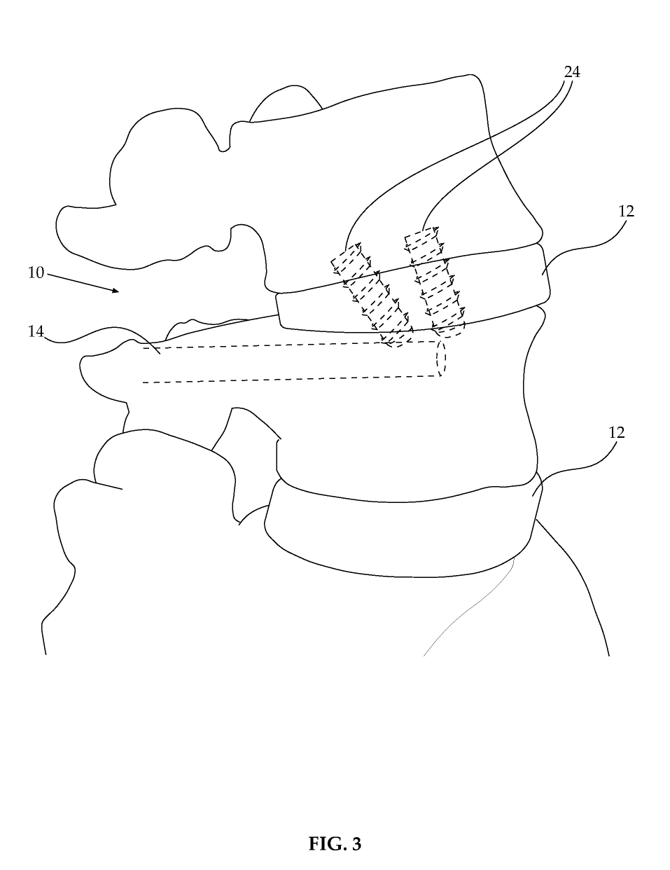

As seen in FIG. 1, a guide wire 18 is inserted through one of the pedicles 20 and passes in a curved trajectory through one of the vertebrae 22 (e.g., L4) and into the intervertebral disc 12. (In another option, wire 18 stops at the first endplate and only the fastener goes through the endplates and the intervertebral disc.) This can be done by a cutting guide wire, curved cannula or other option. Guide wire 18 may pass through intervertebral disc 12 into the adjacent vertebra 23 (e.g., L5). This forms a curved path for insertion of an anterior mechanical supportive fastener 24, which in this embodiment is a threaded fastener 24 (FIG. 2). Threaded fasteners 24 are screwed into the bone by means of a flexible screwdriver 26 (FIG. 2) which is guided along guide wire 18 or the curved path formed by guide wire 18. The flexible screwdriver 26 transfers turning torque to the threaded fasteners 24 in curved geometries. Alternatively, threaded fasteners 24 can be provided with a flexible shaft 28 (FIG. 1) above the threaded portion, and the flexible shaft 28 follows the arcuate path of guide wire 18 out past the pedicles 20. The flexible shaft 28 has a screwdriver slot 30 for inserting a screwdriver (not shown) and turning flexible shaft 28 with the screwdriver screws threaded fastener 24 into the bone. Flexible shaft 28 thus serves as an elastic joint for transferring turning torque to the threaded fastener 24. FIG. 3 illustrates the system after removal of the screwdriver and/or flexible shaft and guide wire 18. The anterior mechanical supportive fasteners 24 secure vertebrae 22 to 23 through intervertebral disc 12, and may be used to secure artificial anterior supports as well (cage and artificial disc).

In one embodiment, pedicle screws 14, having polyaxial swivel heads 16 (seen in FIG. 2 and omitted for simplicity in FIGS. 1 and 3), are fixed in the vertebrae. The pedicle screws 14 may be connected to each other with a connecting rod 15 (not shown in FIGS. 1-3 but seen in the embodiment of FIGS. 4-5) affixed to the polyaxial swivel heads (or fixed head) 16.

In the embodiment of FIGS. 4-5, a guide wire is inserted through one of the pedicles and passes in a curved trajectory through one of the vertebrae and into the intervertebral disc, as described above with reference to FIGS. 1-3. The vertebrae are secured with one or more expandable fasteners 32. Expandable fasteners 32 have an expandable portion 34 (similar to a molly bolt) which is contracted to a narrow profile during insertion of fastener 32 into the bone and is then expanded outwards to the configuration seen in FIGS. 4-5 by turning fastener 32.

Fastener 32 may be turned as above with a flexible screwdriver. FIG. 4 shows another option of turning fastener 32. In this option, a flexible shaft 36 is placed between the end of the shaft of pedicle screw 14 and the head of fastener 32. By turning the pedicle screw 14, flexible shaft 36 transmits the turning torque to expandable fastener 32 so as to expand outwards expandable portion 34. In the embodiment of FIG. 5, expandable fastener 32 does not have flexible shaft 36, and the expandable portion 34 is expanded outwards by turning expandable fastener 32 with a flexible screwdriver (not shown) or by pushing or pulling with an appropriate tool.

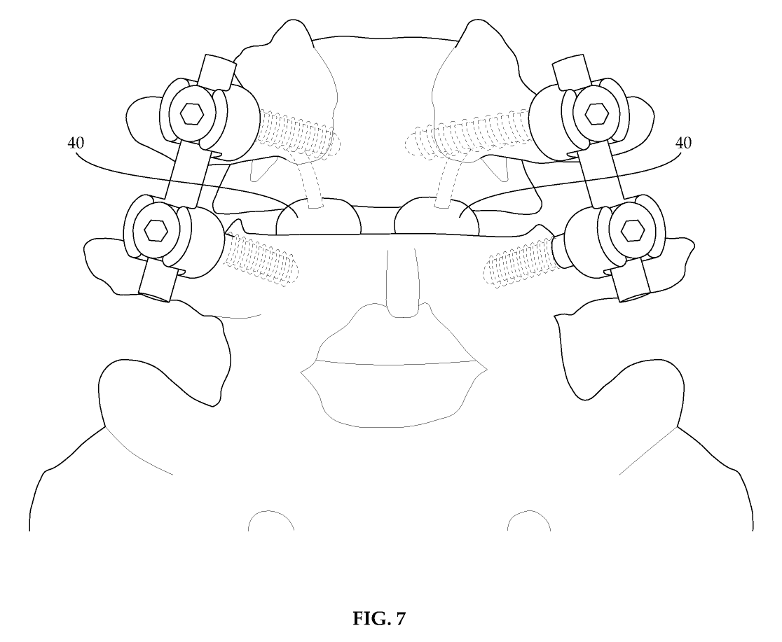

Reference is now made to FIGS. 6 and 7, in which the anterior mechanical supportive fastener is an injectable fastener 40. Injectable fastener 40 has an expandable portion that is fillable and expandable outwards with a fluid, such as but not limited to, air, water, polymer, curable elastomeric material (e.g., a bi-component material that cures with chemical reaction or heating) and other suitable materials that may be injected with a syringe 42 (FIG. 6) and flow to the expandable portion of injectable fastener 40 through the curved trajectory made by the guide wire as above.

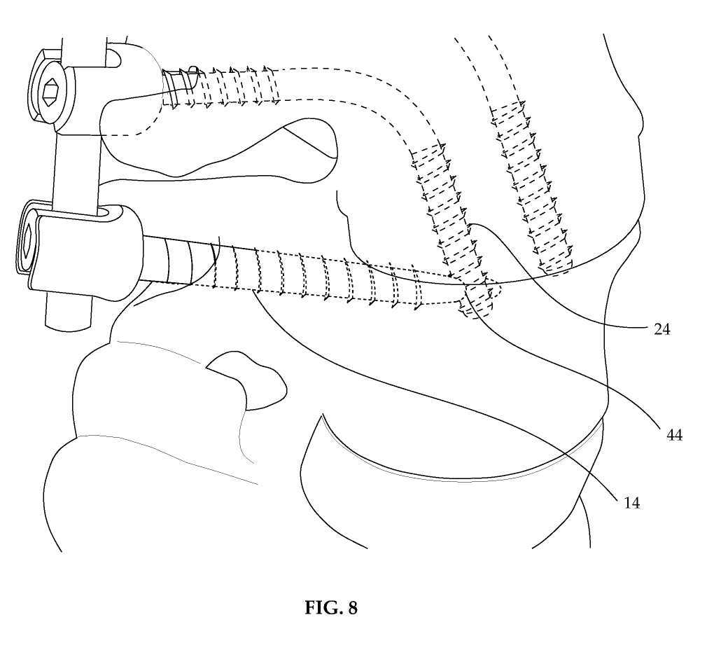

Reference is now made to FIG. 8, which illustrates the mechanical fastener of FIGS. 1-3 connected at the adjacent level to an anterior fastener. In the illustrated embodiment, fastener 24 has a hole 44 formed at an end thereof and the shaft of pedicle screw 14 passes into and optionally through hole 44. This provides double locking of the mechanical fastener with the pedicle screws and provides a much more rigid construct in PLF.

In accordance with other embodiments of the invention, other options are possible. For example, screws may be inserted in a similar approach to improve the fixation of an interbody fixation cage that was previously inserted in any of the optional approaches described above. As another example, screws may be inserted in a similar approach to fix the endplates of an artificial disc prosthesis that was previously inserted in any of the optional approaches. These screws would penetrate only through one vertebra endplate and fixate the adjacent prosthesis endplate.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A method comprising:
affixing an anterior mechanical supportive fastener to anterior portions of adjacent spinal structure by introducing said fastener from a pedicle of a vertebra and passing the fastener through a curved portion to said anterior portions and fastening the fastener to said anterior portions.

2. The method according to claim 1, comprising introducing said fastener through a pedicle at the posterior portion of the vertebra.

3. The method according to claim 1, wherein said fastener is a threaded fastener and fastening the fastener comprises turning the fastener to tighten said fastener.

4. The method according to claim 1, wherein said fastener is an expandable fastener having an expandable portion and fastening the fastener comprises turning the fastener to expand the expandable portion outwards.

5. The method according to claim 1, wherein said fastener is an injectable fastener having an expandable portion that is fillable and expandable outwards with a fluid and fastening the fastener comprises injecting fluid into said expandable portion to expand said expandable portion outwards.

6. The method according to claim 1, further comprising affixing pedicle screws in at least one of the adjacent vertebrae, and securing a connecting rod between heads of said pedicle screws.

7. The method according to claim 3, wherein said threaded fastener is fastened with a flexible screwdriver.

8. The method according to claim 3, wherein said threaded fastener is fastened with a flexible shaft, which serves as an elastic joint for transferring turning torque to said fastener.

9. The method according to claim 6, further comprising connecting said fastener to said pedicle screw.
